(19) United States
(12) Patent Application Publication
(10) Pub. No.: US 2013/0123835 A1
(43) Pub. Date: May 16, 2013

(54) EMBOLIC PROTECTION DEVICE AND METHOD

(71) Applicant: Boston Scientific Scimed, Inc., Maple Grove, MN (US)

(72) Inventors: James Anderson, Fridley, MN (US); Paul J. Miller, Vadnais Heights, MN (US); Huisun Wang, Maple Grove, MN (US)

(73) Assignee: Boston Scientific Scimed, Inc., Maple Grove, MN (US)

(21) Appl. No.: 13/675,657

(22) Filed: Nov. 13, 2012

Related U.S. Application Data
(60) Provisional application No. 61/559,301, filed on Nov. 14, 2011.

(51) Int. Cl. A61F 2/00

(52) U.S. Cl. CPC A61F 2/00
USPC 606/200

(57) ABSTRACT

An embolic protection device adapted for placement in a left subclavian artery, a right subclavian artery and to cover a left and a right carotid artery, the device having a reduced state and an expanded state, the device comprising an expandable support structure, the expandable support structure comprising a frame, the frame further comprising a porous material, the frame defining the circumference of the porous material in the expanded state.

### CROSS-REFERENCE TO RELATED APPLICATIONS

This application claims priority to U.S. Patent Provisional Application No. 61/559,301 filed Nov. 14, 2011, the entire contents of which are hereby incorporated herein by reference.

### BACKGROUND OF THE INVENTION

The present invention generally relates to embolic protection devices and methods of making and using the same.

Heart disease is a major problem in the United States and throughout the world. Conditions such as atherosclerosis result in blood vessels becoming blocked or narrowed. Aortic valve stenosis (AVS) is a disease of the heart valves in which the opening of the aortic valve is narrowed.

Minimally invasive endovascular aortic arch and valve procedures such as transcatheter aortic valve implantation (TAVI) have become a therapeutic option for patients with severe symptomatic aortic stenosis. TAVI is a procedure that involves implantation of a collapsible prosthetic valve using a catheter-based delivery system. This type of prosthesis can be inserted into the patient through a relatively small incision or vascular access site, and can be implanted on the beating heart without cardiac arrest.

Complications of this procedure include embolization of plaque or thrombus. Embolization can occur from the valve during balloon valvuloplasty and valve deployment or embolization of aortic atheroma can occur during device passage.

Embolizations can be carried downstream to lodge elsewhere in the vascular system. This is particularly problematic in both the left and the right carotid arteries. Such emboli can be extremely dangerous to the patient, capable of causing severe impairment of the circulatory system. Depending on where the embolic material is released, a heart attack or stroke could result, or in the event peripheral circulation is severely compromised, the amputation of a limb may become necessary. Thrombus formation can be particularly problematic in structural heart interventional procedures, particularly in minimally invasive heart valve placement procedure and TAVI procedures.

Cerebral embolism or stroke is the sudden blocking of an artery by a thrombus or clot, or other foreign material which is carried to the site of lodgment via blood flow. Cerebral embolism is one of the major complications of transcatheter structural heart procedures or minimally invasive structural heart procedures.

A number of devices, termed embolic protection devices, have been developed to filter out this debris and reduce the risk of cerebral embolism.

Conventional embolic protection devices are used mainly during the carotid vascular interventional procedure whereas the risk of a thrombus embolism is due to carotid vascular angioplasty or stenting.

There remains a need in the art for an embolic protection device that provides effective protection during a transcatheter aortic valve implantation procedure, but also can be used for an extended protection from thrombus embolism after the procedure.

These and other aspects, embodiments and advantages of the present disclosure will become immediately apparent to those of ordinary skill in the art upon review of the Detailed Description and Claims to follow.

### SUMMARY OF THE INVENTION

In one embodiment, the present invention relates to an embolic protection device adapted for placement in a left subclavian artery, a right subclavian artery and to cover a left and a right carotid artery, the device having a reduced state and an expanded state, the device comprising an expandable support structure, the expandable support structure comprising an expandable frame, the expandable frame further comprising a porous material, the frame defining the circumference of the porous material in the expanded state.

### BRIEF DESCRIPTION OF THE DRAWINGS

FIG. 1 is a side perspective view of one embodiment of an embolic protection device according to the invention.

FIG. 2 is a side perspective view of alternative embodiment of an embolic protection device according to the invention.

FIG. 3 is a side perspective view of alternative embodiment of an embolic protection device according to the invention.

FIG. 4A is a side view of one embodiment of an embolic protection device delivered via the right subclavian artery and shown disposed in the right and left subclavian arteries and through the brachiocephalic and the aortic arch.

FIG. 4B is a top down view taken from FIG. 4A.

FIG. 4C is a side view of an embolic protection device similar to that shown in

FIG. 4A delivered via the left subclavian artery and shown disposed in the right and left subclavian arteries and through the brachiocephalic and the aortic arch.

FIG. 5A is a side view of an alternative embodiment of an embolic protection device delivered via the right subclavian artery and shown disposed in the right and left subclavian arteries and through the brachiocephalic and the aortic arch.

FIG. 5B is a side view of an embolic protection device similar to that shown in FIG. 5A delivered via the left subclavian artery and shown disposed in the right and left subclavian arteries and through the brachiocephalic and the aortic arch.

FIG. 6A is an end view of an embodiment of an embolic device shown in an expanded state.

FIG. 6B is an end view of an embolic protection device similar to that shown in FIG. 6A in a partially closed state.

FIG. 6C is an end view of an embolic protection device similar to that shown in FIGS. 6A and 6B in a fully closed state.

FIG. 6D is an alternative embodiment of the embolic protection device in a fully closed state.

### DETAILED DESCRIPTION OF THE INVENTION

While embodiments of the present disclosure may take many forms, there are described in detail herein specific embodiments of the present disclosure. This description is an exemplification of the principles of the present disclosure and is not intended to limit the disclosure to the particular embodiments illustrated.

In one aspect the present invention relates to an embolic protection device for placement in the right and left subclavian arteries and extending therebetween in the brachiocephalic artery and in the aortic arch and covering the left and right carotid arteries. The device can be delivered in a catheter delivery device in a small diameter configuration and then expanded to a larger diameter configuration once in position in the arteries.

In one embodiment, the device includes an expandable support structure, the expandable support structure comprising a frame, the frame further comprising a porous material, the frame defining the circumference of the porous material in the expanded state.

The porous material may in any suitable form which has a pore size small enough to divert emboli from the left and right carotid arteries but large enough to maintain the patency of blood flow through the device.

Examples of suitable porous constructions include membranes, braids, weaves, roves, etc. The size of the openings in the material is suitably between about 50 microns and about 400 microns.

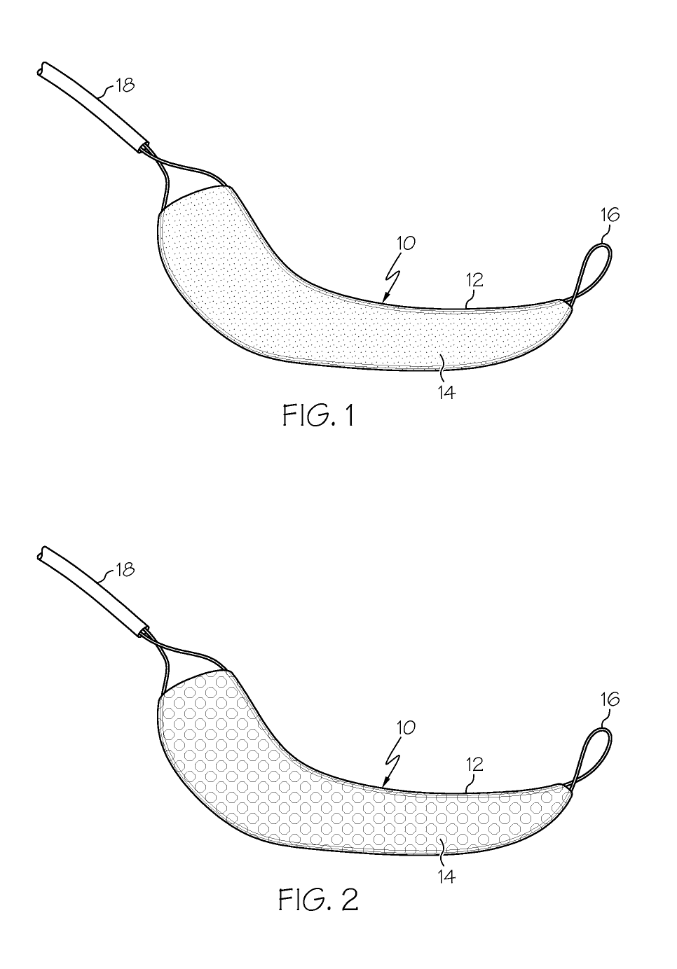

Turning now to the drawings, FIG. 1 illustrates one embodiment of an embolic protection device 10 including an expandable frame 12 and a porous material 14, the circumference of which is defined by the frame 12. Device 10 further includes a loop 16 which can function as an anchor for securing the device in an artery. Device can be delivered using a catheter delivery system 20 shown partially in FIG. 1. In this embodiment, device 10 includes a porous material 14 in the form of a membrane or sponge, the membrane or sponge having an average pore size of about 50 microns.

FIG. 2 illustrates an alternative embodiment of the embolic protection device 10 including an expandable frame 12 and a porous material 14. Device 10 differs from that shown in FIG. 1 in that the porous membrane or sponge in this case has an average pore size of about 100 microns to about 200 microns. This pore size is advantage in that it allows for better blood flow through the device while still diverting larger emboli from passing therethrough.

The porous material can be formed from any suitable biocompatible polymer including, but not limited to, thermoplastic polymers and thermoplastic elastomeric polymer materials such as polyurethanes, polyether-block-amides and nylons. In one embodiment the porous material is formed from a polyurethane.

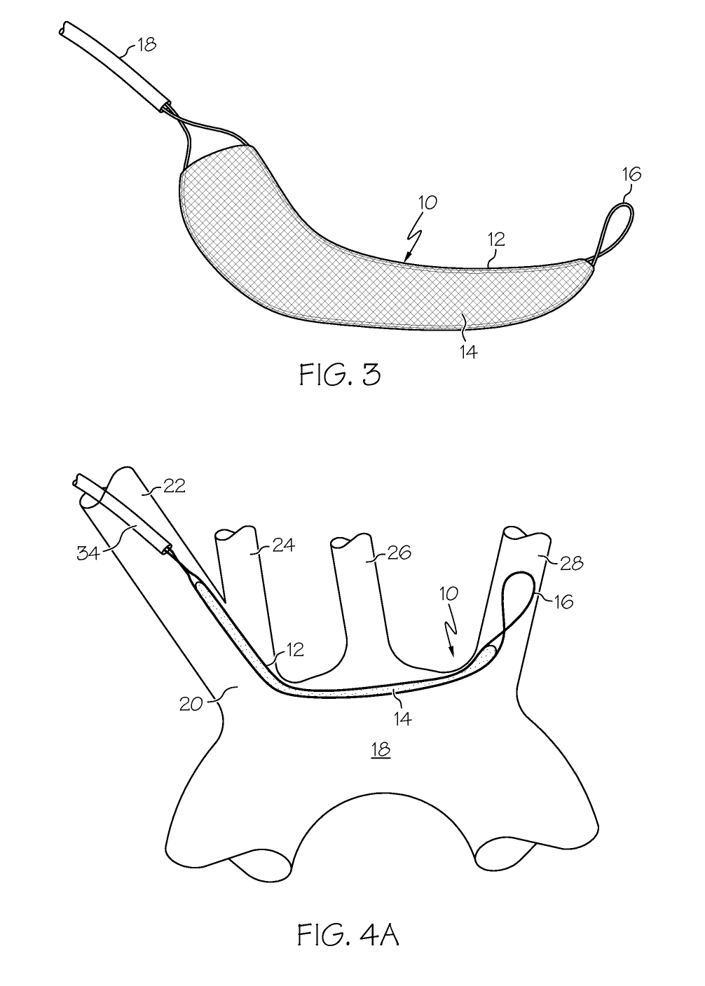

FIG. 3 illustrates an alternative embodiment of an embolic protection device 10 including an expandable frame 12 and a porous material 14 that is in the form of a nitinol mesh. The device can be formed integrally with a nitinol frame, or the mesh can be attached to the frame using welding techniques. Device 10 in this embodiment also has openings from about 100 microns to about 200 microns.

Other shape memory metals and metal alloys and shape memory polymers can be employed in forming frame 12 in any of the embodiments disclosed therein.

Examples of suitable metals or metal alloys include, but are not limited to,

Shape memory polymers including thermoset and thermoplastic polymer can be employed in forming frame 12. Examples of suitable shape memory polymers include, but are not limited to, polyimides, polyether-ether-ketones (PEEK), elastomeric polyurethanes, covalently cross-liked polyurethanes, and so forth.

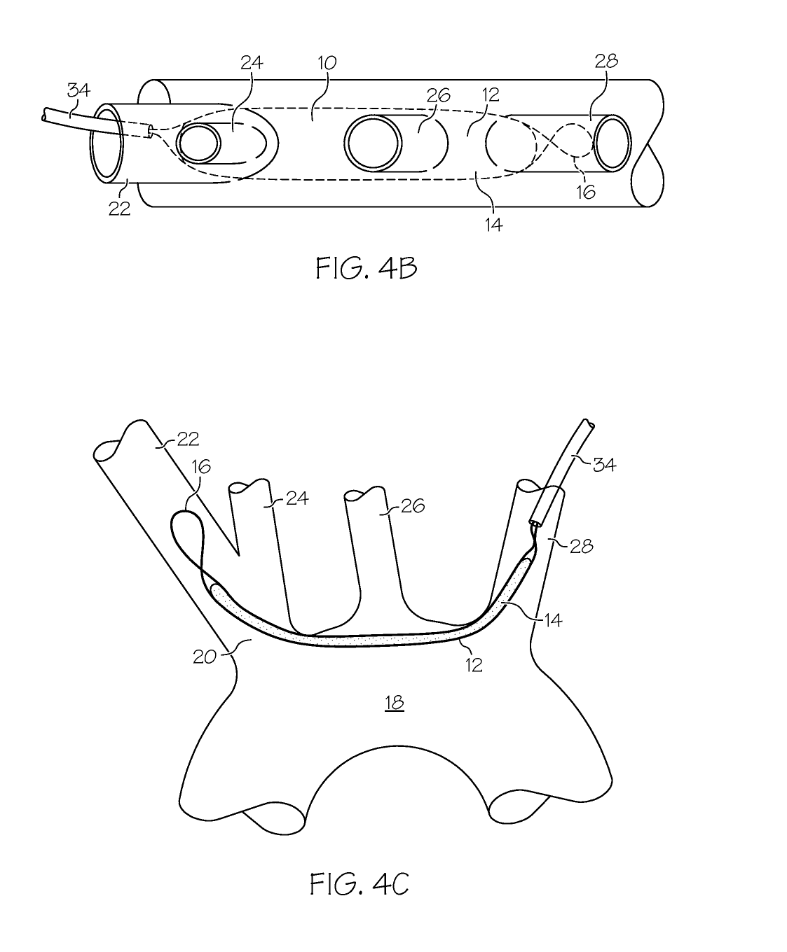

FIG. 4A illustrates an embodiment of an embolic protection device 10 the embodiments of which are described herein above delivered via catheter 34 through the right subclavian artery 22 and into the brachiocephalic artery 20, the aortic arch 18 and the left subclavian artery 28 wherein the device is expanded and catheter 34 can be removed. Device 10 further includes an expandable anchoring means 16 which can be expanded to anchor the device 10 in the left subclavian artery 28. In this embodiment, anchoring means 16 at the distal end of the device which is in the form of a loop which can be integrally formed with frame 12 of device 10. Of course the loop can be formed separately from the device 10 and attached to the frame 12 using any suitable method such as fusion or laser welding. Device 10 covers the right carotid artery 24 and left carotid artery 26 and diverts emboli therefrom.

FIG. 4B is a top down view taken from FIG. 4A.

FIG. 4C illustrates a device similar to that shown in FIGS. 4A and 4B delivered via the left subclavian artery 34.

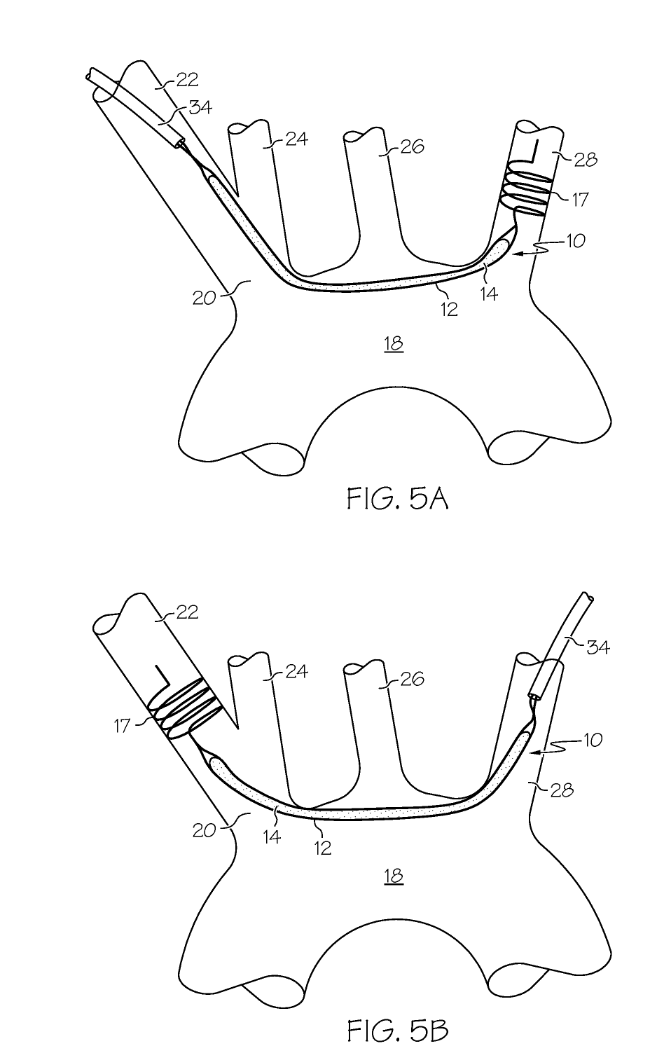

FIG. 5A is an alternative embodiment of an embolic protection device similar to that shown in FIGS. 4A-4C but having an expandable anchoring means 17 at the distal end of the device in the form of a coil or spring which contacts the artery wall, in this case the wall of the left subclavian artery 28. In this embodiment the device is shown delivered via the right subclavian artery 22.

FIG. 5B is an embolic protection device similar to that shown in FIG. 5A delivered via the left subclavian artery 28.

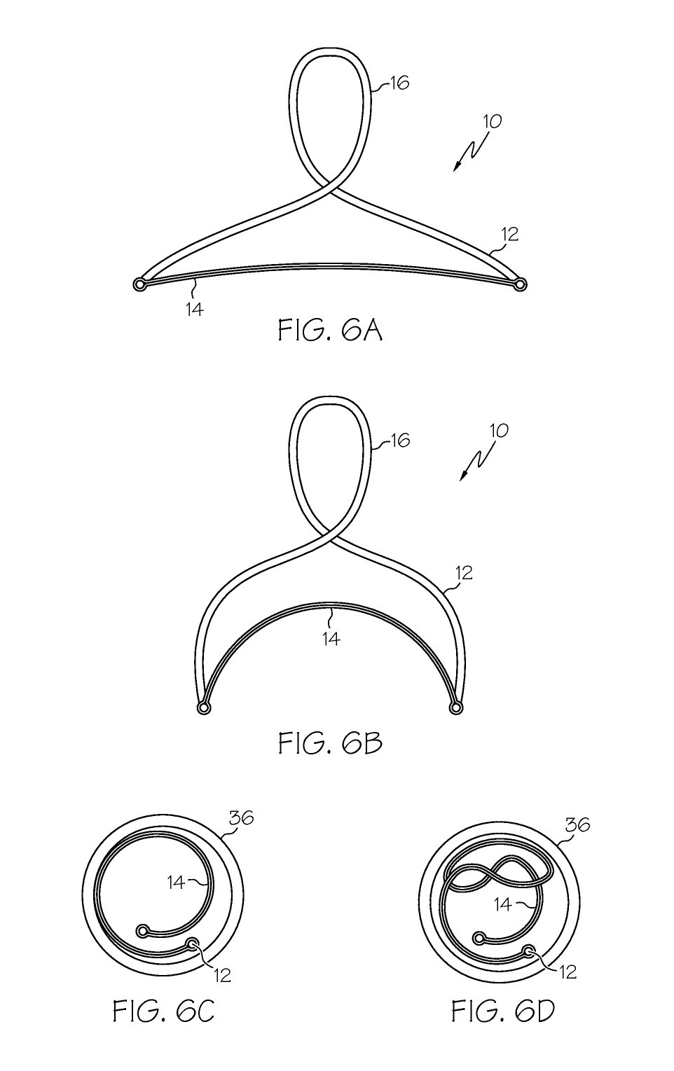

FIGS. 6A-6C illustrate a retrieving sequence for removal of the device from the body of a patient.

FIG. 6A is an end view of an embolic protection device 10 shown in an expanded state.

FIG. 6B is an end view of the device 10 in a partially closed state. Either side of frame 12 rolls toward the longitudinal axis of the device 10.

FIG. 6C is an end view of the device 10 in a fully closed state shown within a retrieving catheter 36. While the majority of emboli are deflected from the device, any emboli which are captured therein may be removed from the artery.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference in their entirety into the specification to the same extent as if each individual publication, patent or patent application was specifically and individually indicated to be incorporated herein by reference. Citation or discussion of a reference herein shall not be construed as an admission that such is prior art.

1. An embolic protection device adapted for placement in a left subclavian artery, a right subclavian artery and to cover a left and a right carotid artery, the device having a longitudinal axis, a reduced state and an expanded state, the device comprising an expandable support structure, the expandable support structure comprising a frame, the frame further comprising a porous material, the frame defining the circumference of the porous material in the expanded state.

2. The embolic protection device of claim 1 wherein said porous material comprises openings of about 50 microns to about 400 microns.

3. The embolic protection device of claim 1 wherein the frame of the device rolls toward the longitudinal axis in the reduced state resulting in a closed structure.

4. The embolic protection device of claim 1 comprising a distal end, a proximal end and a central portion, the device configured and arranged so that when expanded in a patient's body, the distal end of the device is disposed in the left subclavian artery, the proximal end of the device is disposed in the right subclavian artery and the central portion of the device covers the right carotid and left carotid arteries.

5. The embolic protection device of claim 1 comprising a distal end, a proximal end and a central portion, the device configured and arranged so that when expanded in the arteries, the proximal end of the device is disposed in the left subclavian artery, the distal end of the device is disposed in the right subclavian artery and the central portion of the device covers the right carotid and left carotid arteries.

6. The embolic protection device of claim 1 wherein said frame further comprises anchoring means at least at one end of said frame.

7. The embolic protection device of claim 6 wherein said anchoring means is a loop anchor defined by said frame.

8. The embolic protection device of claim 6 wherein said anchoring means is an expandable coil connected to said frame of said device.

9. The embolic protection device of claim 1 wherein said frame comprises a shape memory metal or a shape memory polymer.

10. The embolic protection device of claim 9 wherein frame is a shape memory metal, said shape memory metal is nitinol.

11. The embolic protection device of claim 1 wherein said porous material comprises a polymer.

12. The embolic protection device of claim 11 wherein said polymer is a polyurethane.

13. The embolic protection device of claim 1 wherein said porous material comprises a membrane, weave, mesh, braid or rove.

14. The embolic protection device of claim 1 wherein said porous material comprises openings of about 50 microns to about 200 microns.

15. The embolic protection device of claim 1 wherein said porous material is fastened to said expandable frame with an adhesive.

16. The embolic protection device of claim 1 wherein said porous material is fastened to said expandable frame by welding.

17. The embolic protection device of claim 1 wherein said expandable frame and said porous material are formed from the same material.

18. The embolic protection device of claim 17 wherein said material is a shape memory metal.

19. The embolic protection device of claim 17 wherein said shape memory metal is nitinol.

20. The embolic protection device of claim 18 wherein said porous material is a wire mesh having a pore size of about 100 microns to about 200 microns.

21. The embolic protection device of claim 1 wherein said expandable frame and said porous material are formed from a different material.
